(19) United States
(12) Patent Application Publication
(10) Pub. No.: US 2026/0241197 A1
(43) Pub. Date: Aug. 20, 2026

(54) LIGHT THERAPY DEVICES, SYSTEMS, METHODS

(71) Applicant: Lumebox LLC, San Ramon, CA (US)

(72) Inventors: Hua Liu, Irvine, CA (US); Vivian Chen, San Ramon, CA (US)

(21) Appl. No.: 19/544,889

(22) Filed: Feb. 19, 2026

Related U.S. Application Data
(60) Provisional application No. 63/761,061, filed on Feb. 20, 2025.

(51) Int. Cl. A61N 5/06; A61N 5/00

(52) U.S. Cl. CPC A61N 5/0616; A61N 2005/005; A61N 2005/0626; A61N 2005/0632; A61N 2005/0645; A61N 2005/0652; A61N 2005/0659; A61N 2005/0663

(57) ABSTRACT

A light therapy device can include a front portion. The front portion can include a flexible substrate, a plurality of channels positioned on an exterior surface of the flexible substrate, and a base strip embedded in the flexible substrate. The base strip can be configured to house a plurality of light-emitting diodes (LEDs). The base strip can include one or more materials having a harder elasticity than the flexible portion. The front portion can include a heat sink positioned on a rear of the base strips. The light therapy device may include a rear portion. The rear portion can include a rear surface, a plurality of support strips position in an interior of the rear portion, and a plurality of channels positioned in the rear surface. The heat sink can be configured to enable the transfer of heat generated in the plurality of LEDs to the base strips.

### CROSS REFERENCE

Any and all applications for which a foreign or domestic priority claim is identified in the Application Data Sheet as filed with the present application are hereby incorporated by reference under 37 CFR 1.57. This application claims priority to U.S. provisional patent application No. 63/761,061, filed on February 20, 2025, the disclosure of which is incorporated by reference herein in its entirety.

### BACKGROUND

### Field

The present disclosure relates to light therapy devices (also referred to as phototherapy devices), including devices that use redlight therapy (RLT), to provide therapeutic light to a user’s skin.

### Certain Related Art

Light therapy devices can be used in different applications, including treatment of various medical conditions, improvement of skin appearance, addressing aesthetic concerns, and/or the like.

### SUMMARY OF CERTAIN FEATURES AND ASPECTS

Existing light therapy devices fail to provide efficient and/or effective light therapy. Accordingly, a need exists for an improved light therapy device.

One concern relates to light therapy devices that can operate at elevated temperatures (e.g., temperature(s) that make the light device uncomfortable to use, hold, etc. and/or that make the device harmful or dangerous to set down, such as on a towel). Light therapy devices can include lighting features such as Light-Emitting Diodes (LEDs). LEDs generate heat as they operate. Materials such as foam, neoprene, and/or the like, when used in a light therapy device, can absorb the heat that is generated in LEDs. As a result, the temperature of such devices increases during use, making them uncomfortably hot to contact the skin of a user. In some embodiments, the devices of the present disclosure can include a heat sink and/or can utilize double injection molding of a soft (e.g., silicone-type) material with hard (e.g., plastic) strips to facilitate the transfer of heat. Advantageously, this allows such devices to operate at a lower temperature, thereby making them more comfortable for the user.

Another concern relates to the safety and efficacy of light therapy devices. Devices that use flammable materials can pose safety concerns. Such devices need to be configured to operate with lower electrical power (e.g., lower Wattage) to lower the risk of flammability. Consequently, this affects the efficacy of these devices since they operate with lower electrical power. In some embodiments, the devices of the present disclosure include a heat sink and/or can utilize double injection molding of a soft (e.g., silicone-type) material with hard (e.g., plastic) strips to control the temperature of the device. Advantageously, this allows the use of more electrical power and/or more powerful LEDs, thereby providing high irradiance while maintaining flexibility in the device.

Another concern relates to the size of a light therapy device. As a light therapy device operates, the light features (e.g., LEDs) positioned in the device generate heat. A cooling mechanism can thus be included in such devices. Devices that use large cooling mechanisms (e.g., fans) require boxes and/or large housings to fit the cooling mechanisms, thereby increasing the size of the device. Large light therapy devices can have limited use and/or be uncomfortable. For example, a user may be uncomfortable lying on a large device to treat their back. In some embodiments, the devices of the present disclosure can use a combination of features (e.g., heat sinks, hard plastic support strips, etc.) to create a light therapy device that includes internal cooling features and/or does not require external cooling. Advantageously, this results in a device that is small, flexible, and comfortable to use on different body parts.

Another concern relates to providing substantially uniform and/or continuous coverage throughout the body part that is to receive light therapy. If a light therapy device is too large or bulky to flex over a body part, the light supplied to that body part will not be uniform and/or continuous, so the treatment will be sub-optimal and/or ineffective. In some embodiments, the devices of the present disclosure provide a continuous network of light features (e.g., LEDs) that can provide continuous coverage over the body part. For example, certain variants inhibit or avoid the presence of “dead spots” in which substantially less light is delivered. In some implementations, the devices of the present disclosure can use a combination of features (e.g., using hard plastic and soft rubberized material) that enable such devices to flex and adjust to the contour of the body, thereby facilitating a uniform coverage over the body part.

The present disclosure relates to light therapy devices that address one or more of the above-described concerns or other concerns.

Some embodiments of the light therapy devices disclosed herein are configured to maintain flexibility while also providing increased LED power and/or irradiance levels (compared to one or more of the devices described above). In some embodiments, the light therapy devices of the present disclosure can include LEDs embedded in one or more base strips (e.g., strips of hard plastic). The base strips can be embedded in a flexible substrate, such as a silicone substrate. Some embodiments are formed by double (also called “two part”) injection molding of the base strips into the substrate corresponding to the front portion and/or double injection molding of support strips in a flexible substrate corresponding to the back portion. The substrates of the front portion can include grooves between adjacent base strips. The grooves can aid with flexibility. Advantageously, one or more of these features can provide strength to the devices of the present disclosure while maintaining softness and flexibility.

The light therapy devices of the present disclosure can include one or more bases. In various implementations, the bases are strips, so can be referred to as base strips herein, but the bases are not limited to strips. The bases (e.g., base strips) can include one or more LEDs. In some embodiments, the light therapy devices of the present disclosure can include a total of at least 20 LEDs, 30 LEDs, 40 LEDs, 50 LEDs, fewer than 20 LEDs, more than 50 LEDs, or any other number of LEDs depending on the user’s need. The LEDs can be spaced in proximity to one another, such that a continuous or substantially continuous light coverage can be provided to the user’s skin. The LEDs can be communicatively connected to one another (e.g., with one or more wires) and/or to an external power source (e.g., a battery pack, a plug, etc.).

Some embodiments of light therapy devices of the present disclosure are configured to efficiently transfer the heat that is generated while such devices are operated. As discussed further below, heat sinks (e.g., aluminum plates) can be positioned adjacent to (e.g., behind) the base strips. Some embodiments of the devices disclosed herein can include a groove and/or a channel between each strip of LEDs. Advantageously, the grooves and/or channel can provide a passage for the heat to vent out from the device, thereby reducing the temperature perceived by the user’s skin while allowing a high level of irradiance and flexibility. In various embodiments, the light therapy device is configured to vent heat passively and/or does not include a cooling fan.

In some embodiments, the light therapy devices of the present disclosure include a front portion and a rear portion. The front portion can include a plurality of LEDs and heat sinks. The rear portion can include a plurality of supports. In some embodiments, the supports can be in the form of strips and can be referred to as support strips. The support strips can be embedded in a flexible substrate (e.g., silicone). The heat sinks can be configured to engage with a corresponding support strip. The heat sinks and support strips can form a pin and slot configuration. For example, the heat sinks can include a pin that can mate with or be positioned in a slot in the support strip. In some embodiments, the support strips direct the flow of heat from the heat sinks to the sides of the light therapy device. The support strips can also inhibit the heat from spreading through the silicone substrate.

Some embodiments of the present technology include a light therapy device configured to provide a thin profile. Devices with a thin profile can be used in more applications and/or be more comfortable. For example, such devices can be used in applications that provide light therapy to the back of a user, allowing the user to lie on the device without excessive discomfort. In one example, the light therapy devices of the present disclosure include a thickness of about 10.2 millimeters (mm). In some embodiments, the light therapy devices of the present disclosure include thin lenses positioned over LED bulbs. These lenses can be configured to diffuse the light effectively and substantially evenly while maintaining a thin profile.

In some embodiments, the techniques described herein relate to a light therapy device configured to emit light to a body part of a user, the light therapy device including: a front portion, the front portion including: a first flexible substrate; a plurality of channels positioned on an exterior of the flexible substrate; a base strip embedded in the flexible substrate, the base strip being harder than the flexible substrate; a plurality of lighting features in the base strip; and a heat sink positioned adjacent to the plurality of lighting features, the heat sink positioned on an interior side of the front portion; and a rear portion, the rear portion including: a second flexible substrate; a plurality of support strips positioned in an interior of the rear portion, the plurality of support strips being harder than the flexible substrate of the rear portion; and a plurality of channels positioned on the exterior of the rear portion; wherein the heat sink is configured to transfer heat generated by the plurality of lighting features to the plurality of support strips.

In some embodiments, the techniques described herein relate to a light therapy device, wherein the flexible substrate of the front portion and the flexible substrate of the rear portion include silicone.

In some embodiments, the techniques described herein relate to a light therapy device, wherein the base strip includes PC-ABS.

In some embodiments, the techniques described herein relate to a light therapy device, wherein the rear portion is manufactured by double injection molding of the flexible substrate and the plurality of support strips.

In some embodiments, the techniques described herein relate to a light therapy device, wherein the heat sink is an aluminum plate.

In some embodiments, the techniques described herein relate to a light therapy device, wherein the light therapy device is configured to emit lights having a wavelength and frequency corresponding to red light.

In some embodiments, the techniques described herein relate to a light therapy device, wherein the light therapy device is configured to emit light including wavelengths associated with infrared.

In some embodiments, the techniques described herein relate to a system including the light therapy device and a controller.

In some embodiments, the techniques described herein relate to a system, wherein the controller includes a control interface and a processor to control an operation of the light therapy device.

In some embodiments, the techniques described herein relate to a system, wherein the controller is configured to control the operation including: an activation of the light therapy device, a predetermined time to deactivate the light therapy device, or a type of light that the light therapy device is configured to emit.

In some embodiments, the techniques described herein relate to a method of providing light therapy to a body part of a user, the method including: positioning a light therapy device over the body part, the light therapy device having a flexible body, the light therapy device including a first end and a second end, the first end including a first aperture and the second end including a second aperture; positioning a band through the first aperture and the second aperture to form a loop; securing the loop of the band with the body part of the user; activating the light therapy device; enabling the light therapy device to emit light to the body part of the user, the light corresponding to a predetermined frequency; and deactivating the light therapy device after a passage of a predetermined time.

In some embodiments, the techniques described herein relate to a method, wherein activating the light therapy device includes providing one or more commands to a controller through a control interface.

In some embodiments, the techniques described herein relate to a method, wherein a step of deactivating the light therapy device is performed by a controller after the passage of the predetermined time.

In some embodiments, the techniques described herein relate to a method, wherein the band is an elastic band including a first end and a second end.

In some embodiments, the techniques described herein relate to a method, wherein securing the loop of the band with the body part includes coupling the first end and the second end of the elastic band with a hook-and-loop system.

In some embodiments, the techniques described herein relate to a light therapy device, including: a body including a flexible portion and a hard portion, the body configured to conform with a body part of a user; a plurality of channels positioned on the flexible portion, the plurality of channels configured to enable the body to flex; and a plurality of light features configured to emit light to a body part of the user, the plurality of light features arranged to form an array of lights including a series of rows and columns, the rows arranged along a lateral orientation of the light therapy device and the columns arranged along a longitudinal orientation of the light therapy device, the array of lights configured to emit light to the body part uniformly.

In some embodiments, the techniques described herein relate to a light therapy device, wherein the array of lights includes ten rows and four columns.

In some embodiments, the techniques described herein relate to a light therapy device, wherein the body includes a front portion configured to mate with a rear portion.

In some embodiments, the techniques described herein relate to a light therapy device, wherein each of the plurality of light features includes a light-emitting diode and a lens, the light-emitting diode configured to emit red light.

In some embodiments, the techniques described herein relate to a light therapy device, including a plurality of heat sinks positioned adjacent to the plurality of light features.

Neither the preceding Summary nor the following Detailed Description and associated drawings purport to limit or define the scope of protection. The scope of protection is defined by the claims.

### BRIEF DESCRIPTION OF THE DRAWINGS

These and other features, aspects, and advantages of the present disclosure are described with reference to the drawings of certain embodiments, which are intended to illustrate certain embodiments and not to limit the invention, in which like reference numerals are used for like features, and in which:

FIG. 1 illustrates a top perspective view of a light therapy device according to one embodiment of the present disclosure.

FIG. 2A illustrates a bottom perspective view thereof.

FIG. 2B illustrates a bottom partially exploded perspective view thereof.

FIG. 2C illustrates an exploded view thereof.

FIG. 3 illustrates a front view thereof.

FIG. 4 illustrates a rear view thereof.

FIG. 5 illustrates a left-side view thereof.

FIG. 6 illustrates a right-side view thereof.

FIG. 7 illustrates a top view of a front portion thereof.

FIG. 8 illustrates a top view of a rear portion thereof.

FIG. 9A illustrates an interior of the front portion of the light therapy device of FIG. 1.

FIG. 9B illustrates an interior of the front portion of the light therapy device, with a plurality heat sinks installed therein, according to some embodiments.

FIG. 10 illustrates an interior of the rear portion of the light therapy device of FIG. 1.

FIG. 11 illustrates an interior of the front portion and rear portion of the light therapy device of FIG. 1.

FIGS. 12A and 12B illustrate a perspective view of the light therapy device of FIG. 1.

FIG. 13 illustrates the light therapy device of FIG. 1 with an external (e.g., handheld) controller.

FIG. 14 illustrates the light therapy device of FIG. 1 with a controller and a band.

FIG. 15 illustrates a block diagram of an exemplary light therapy system.

FIG. 16 illustrates an example of the light therapy device of FIG. 1 in use.

### DETAILED DESCRIPTION OF CERTAIN EMBODIMENTS

The various devices, systems and methods disclosed herein are suited to provide red light therapy and/or other types of light therapy. Advantageously, various embodiments of such devices, systems, and/or methods can allow for providing light therapy to a user’s body part (e.g., face, back, etc.). The embodiments disclosed herein offer one or more advantages and benefits over existing technologies for providing light therapy, such as, for example and without limitation, facilitating an efficient and effective light therapy, providing a device that is flexible (e.g., which can be particularly important to provide a uniform coverage over a body part), taking advantage of materials that do not absorb excessive heat and/or the like. For purposes of summarizing the disclosure, certain aspects, advantages and features of the inventions are described herein. Not necessarily any or all such advantages are achieved in accordance with any particular embodiment of the inventions disclosed herein. No aspects of this disclosure are essential or indispensable.

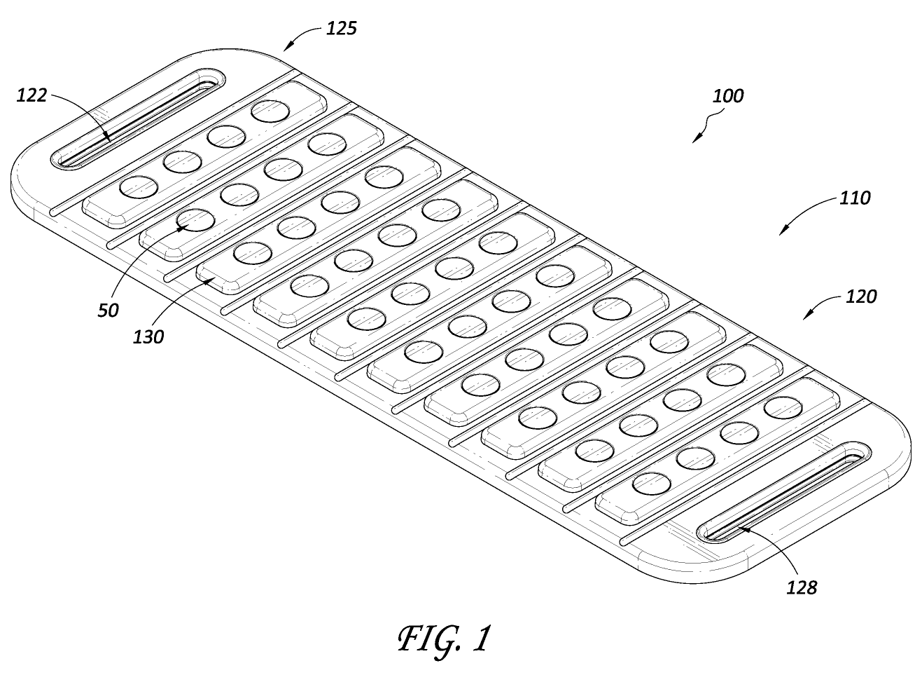

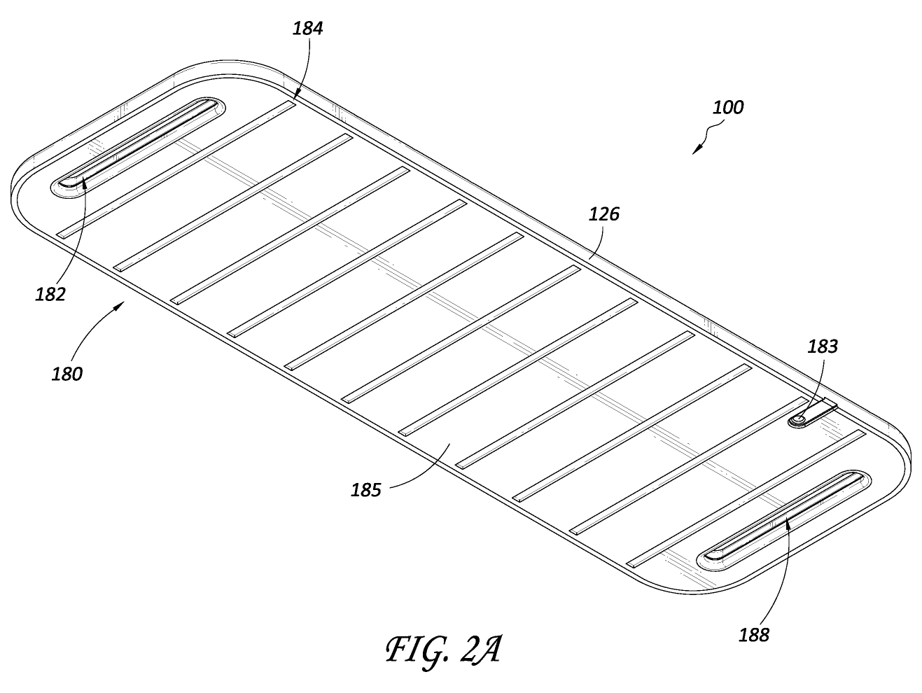

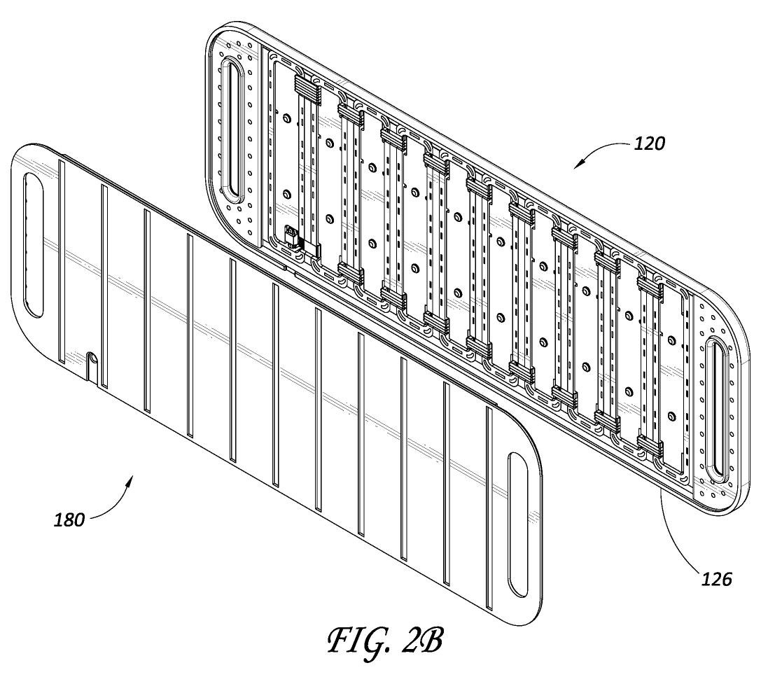

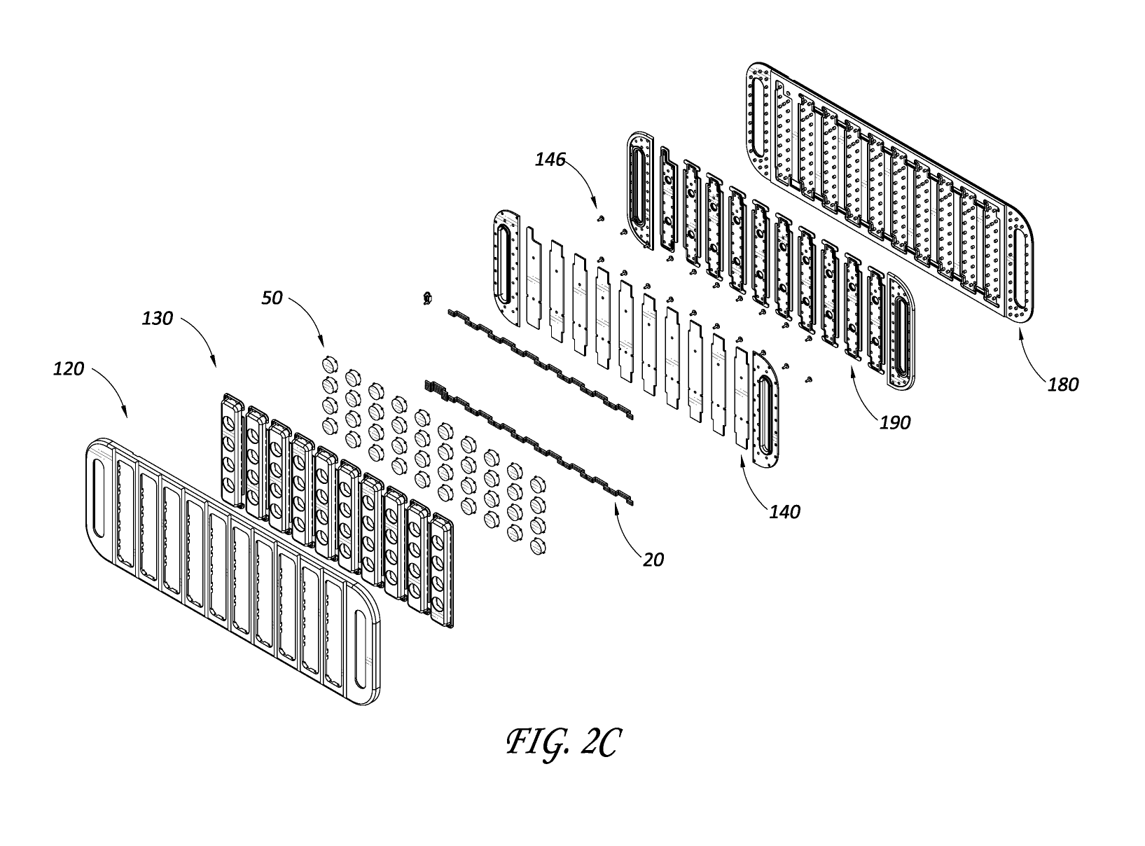

FIG. 1 illustrates a top perspective view of a light therapy device 100 according to one example of the present disclosure. The light therapy device 100 can include a body 110. The body 110 can include a front portion 120, and a rear portion 180 (not illustrated in FIG. 1 but see FIG. 2A). The front portion 120 can mate with the rear portion 180 to form an enclosure. The body 110 can be made from one or more hard materials, soft materials, and/or a combination of hard materials and soft materials. For example, the body 110 can include soft materials capable of elastic deformation and hard materials generally resistant to deformation. In some embodiments, the soft materials of the body 110 can have a modulus of elasticity of about 0.1MPa to about 70MPa, and the hard materials of the body 110 can have a modulus of elasticity of about 1.7GPa to about 3GPa. The body 110 can be manufactured in different sizes and dimensions to accommodate a user’s need. In one example, the body 110 can be manufactured to form a generally rectangular shape with round edges. In some embodiments, the body component 110 can be substantially continuous. For example, the body component 110 can be substantially continuous in a longitudinal direction without any separation. Advantageously, this facilitates providing a substantially continuous distribution of light to a body part of the user. In one example, the body 110 can include a generally rectangular shape having a length of at least about 318mm and a width of less than or equal to about 114mm.

As discussed above, the body 110, including the front portion 120, can be manufactured from a combination of hard and soft materials. For example, the front portion 120 can be manufactured such that it includes a soft portion and a hard portion. In some embodiments, the soft portion of the body 110 can include soft and/or flexible rubberized materials (e.g., thermoplastic elastomer (TPU), silicone, etc.). In some embodiments, the hard portion of the body 110 can include a blend of polycarbonate (PC) and acrylonitrile butadiene styrene (ABS) material (commonly referred to as PC-ABS). In some embodiments, the front portion 120 can include a soft portion 125 (also referred to as a flexible substrate) and a hard portion (e.g., base strip 130) embedded in the soft portion.

The light therapy device 100 can include a plurality of light features, such as a light feature 50 positioned in the base strip 130. The light features can be configured to deliver therapeutic light, as further described below.

The front portion 120 can include a first aperture 122 and a second aperture 128. In some embodiments, the first aperture 122 and/or the second aperture 128 can include an elongate body. The apertures can be configured to receive a band (e.g., strap), as discussed in more detail below.

FIG. 2A illustrates a bottom perspective view of a rear portion 180 of the light therapy device 100. The rear portion 180 can include a first aperture 182 and a second aperture 188. In some embodiments, the first aperture 182 and/or the second aperture 188 can include an elongate body. The first aperture 182 and/or the second aperture 188 can cooperate with and complement the first aperture 122 and the second aperture 128 (see, e.g., FIG. 1) of the front portion 120 to receive a band therethrough.

The rear portion 180 can include a soft portion and a hard portion. For example, the rear portion 180 can include a soft portion (e.g., at a rear surface 185) and a hard portion (e.g., around the periphery of the first aperture 182 and/or the second aperture 188, etc.) embedded in the soft portion.

FIG. 2B illustrates the rear portion 180 separated from the front portion 120 for illustrative purposes. As shown, the front portion 120 can include interior components surrounded by a peripheral edge 126. In some embodiments, the peripheral edge 126 corresponds to the front portion 120 (as illustrated in FIG. 2B). For example, the front portion 120 can include a planar surface and the peripheral edge 126 can extend generally perpendicular to and/or around the planar surface.

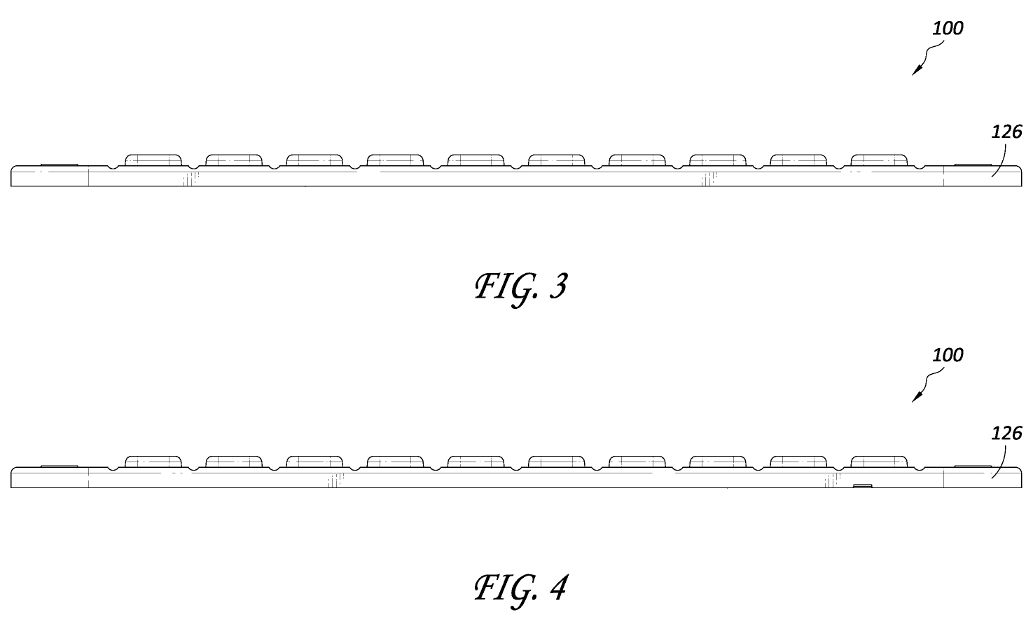

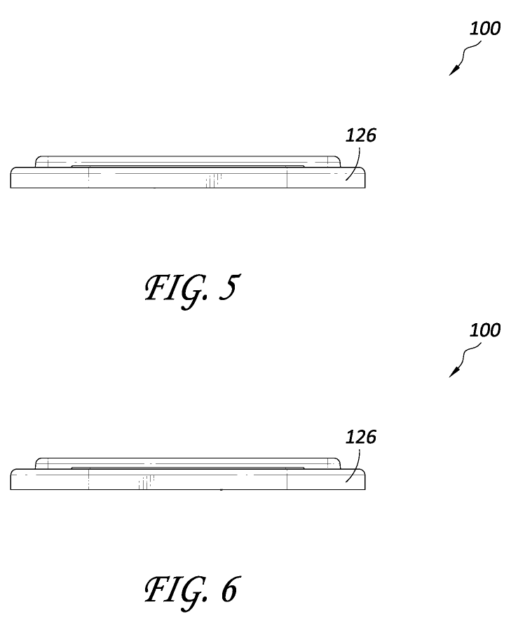

FIGS. 3 and 4 illustrate the front and rear sides of the light therapy device 100 along the length direction. FIGS. 5 and 6 illustrate the left and right sides of the light therapy device 100 along the width direction. The peripheral edge 126 can be seen in FIGS. 3 through 6. The peripheral edge 126 extends around the light therapy device 100. The peripheral edge 126 can include a depth of less than or equal to: about: 5mm, about 6mm, about 7mm, or other values depending on the user’s need. Advantageously, these small values corresponding to the peripheral edge 126 can enable the light therapy device 100 to include a thin body. For example, the light therapy device 100 can have a depth or thickness (corresponding to the peripheral edge 126) that is substantially smaller than its length or width. In one example, the light therapy device 100 can have a length of at least about 318mm, a width of less than or equal to about 114mm, and a thickness of less than or equal to about 6.6mm (or less than or equal to about 10.2mm including the base strips). In certain implementations, a ratio of the device’s length to width is at least about: 2.5, 2.7, 2.9, 3.0, etc. In some examples, a ratio of the device’s length to thickness is at least about: 47.5, 48.2, 49.4, 50.1, etc. In some variants, a ratio of the device’s width to thickness is at least about: 16.9, 17.3, 18.6, 19.5, etc.

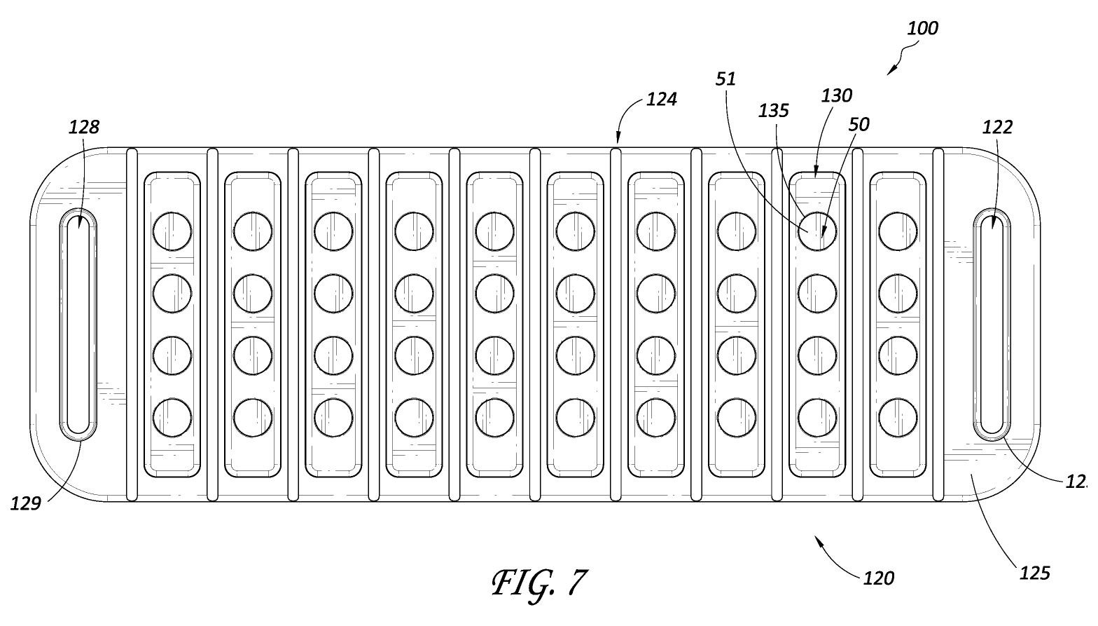

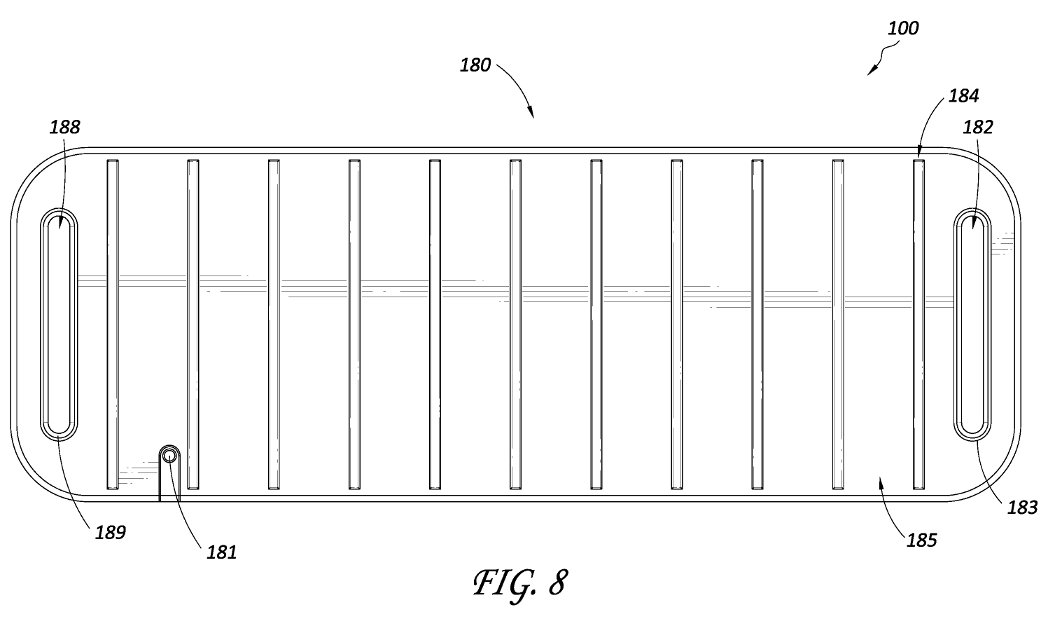

FIG. 7 illustrates a top view of the light therapy device 100. As discussed above, the front portion 120 of the light therapy device 100 can include the first aperture 122 and the second aperture 128. The first aperture 122 and the second aperture 128 of the front portion 120 can cooperate with the corresponding apertures in the rear portion 180 (see, e.g., first aperture 182 and second aperture 188 in FIG. 8) to form an opening (e.g., through hole) on either end of the light therapy device 100. Such opening(s) can be used to secure an elastic band or a belt loop (see, e.g., band 80 in FIG. 14). The first aperture 122 can include a perimeter edge 123, and the second aperture 128 can include a perimeter edge 129. The perimeter edge 123 and/or the perimeter edge 129 can include materials that are generally harder than the soft portion 125, which can help to better secure an elastic band therethrough.

The front portion 120 of the light therapy device 100 can include one or more light features, such as the light feature 50. The one or more light features 50 can be spaced apart and/or spread through the front portion 120.

The light feature 50 can include an illuminating component, such as an LED or other light emitting device. The light feature 50 can be configured to emit lights having a predetermined frequency and/or wavelength of the electromagnetic spectrum. For example, the light feature 50 can be configured to deliver light having a wavelength and/or frequency corresponding to visible red light (e.g., light with a wavelength of between about 620 and about 750 nanometers). In some embodiments, the light feature 50 can be positioned on a heat sink.

The light feature 50 can include a lens 51 that covers the illuminating component. The lens can be configured to diffuse the light emitted from the illuminating component to the body of the user more effectively. The lens 51 can be made from one or more materials that have optical clarity (e.g., Polymethacrylate (PMMA), Polycarbonate (PC), glass, etc.). The lens 51 can be made in a variety of shapes (e.g., convex, flat, dome, etc.) while maintaining a thin profile.

The light feature 50 can be located and/or nested in a housing 135 in the base strip 130. In some embodiments, the light therapy device 100 can include one or more rows of the light features 50 positioned in the base strip 130. For example, the light therapy device 100 can include one row, two rows, three rows, four rows, five rows, six rows, seven rows, eight rows, nine rows, ten rows, or more than ten rows of base strip 130. Some or each row of the base strip 130 can include one or more light features 50. In one example, the light therapy device 100 includes ten rows of the base strip 130, each row including four light features 50. The light features 50 can be configured to form an array of lights. In several embodiments, the array of lights facilitates providing a substantially uniform distribution of light to a body part of the user, illuminate a desired amount of irradiance to a body part of the user, and/or provide other benefits.

The light features 50 can be located in the light therapy device 100 in a variety of arrangements. In some embodiments, the light features 50 can be positioned in the light therapy device 100 to form a series of rows and columns. In some embodiments, the light features 50 in each row of the base strip 130 can be aligned or substantially aligned along a lateral axis extending through the center of each of the light features 50 in the base strip 130. In some embodiments, the light features 50 in each column can be aligned or substantially aligned along a longitudinal axis extending through the center of each of the light features 50.

Each row of the base strip 130 can be separated from another row (e.g., a longitudinally adjacent row) of the base strip 130 by a channel 124. In some embodiments, the channel 124 can include an elongated narrow recess or depression (e.g., a groove) in the body of the front portion 120. The separation of base strips 130 by the channel 124 can help with uniform distributions of lights over a body part of the subject, can enhance the flexibility of the light therapy device, and/or can allow the heat generated by the light features 50 to vent out. For example, the channel 124 can separate each row of the light features 50 from one another such that illumination of light features 50 in each row do not overlap with one another. The channel 124 can allow the front portion 120 to flex and/or bend, thereby allowing the front portion 120 to better conform with the contour of the user’s body.

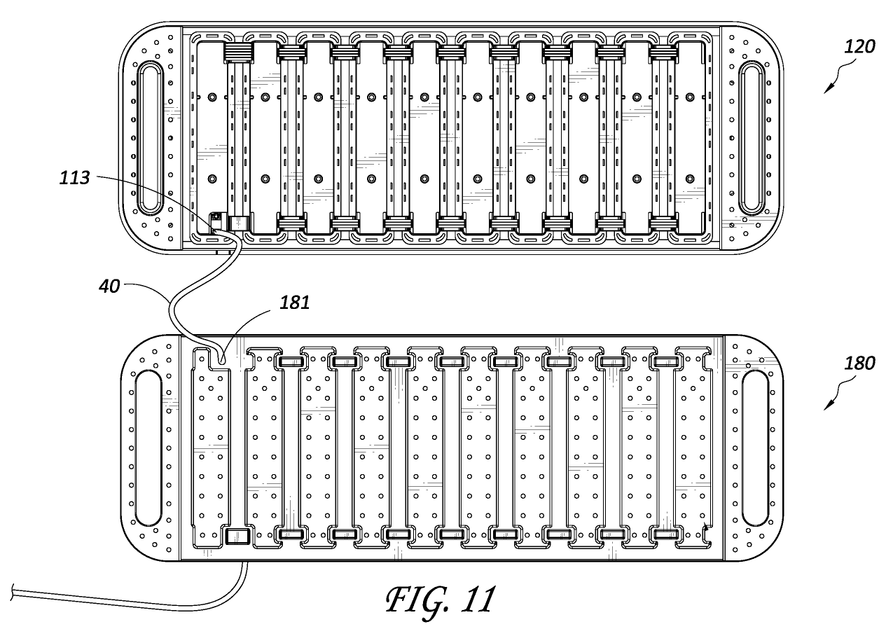

FIG. 8 illustrates the rear portion 180 of the light therapy device 100. As discussed above, the rear portion 180 can include the first aperture 182 and the second aperture 188. The first aperture 182 can include a perimeter edge 183, and the second aperture 188 can include a perimeter edge 189. The perimeter edge 183 and/or 189 can include one or more materials that are generally harder than the rear surface 185. Advantageously, the harder material of the perimeter edges 183 and 189 provide a housing that is more secure to accommodate an elastic band, as illustrated in FIG. 14 and discussed further below. The rear portion 180 can include an aperture 181. The aperture 181 can be configured to allow the passage of a connector 40 therethrough. See connector 40 in FIGS. 11A and 11B.

The rear portion 180 can include one or more channels 184. The channel 184 can be an elongated narrow recess or depression (e.g., a groove) in the body of the rear portion 180. The channels 184 can allow the rear portion 180 to flex and/or bend, thereby allowing the rear portion 180 to conform with the contour of the user’s body. In some embodiments, the channels 184 of the rear portion 180 can be positioned such that they are aligned with a corresponding channel on the front portion 120 (e.g., the channels 124) and/or are positioned between adjacent base strips 130. This arrangement allows the channels 124 of the front portion 120 to cooperate with the channels 184 of the rear portion 180 to facilitate the flexing of the light therapy device 100, thereby enabling the light therapy device 100 to better conform with the curvature of the desired body part. As an example, as shown in FIG. 7 and 8, the channels 184 of the rear portion 180 are aligned (e.g., in the length and/or width direction) with the channels 124 of the front portion 120.

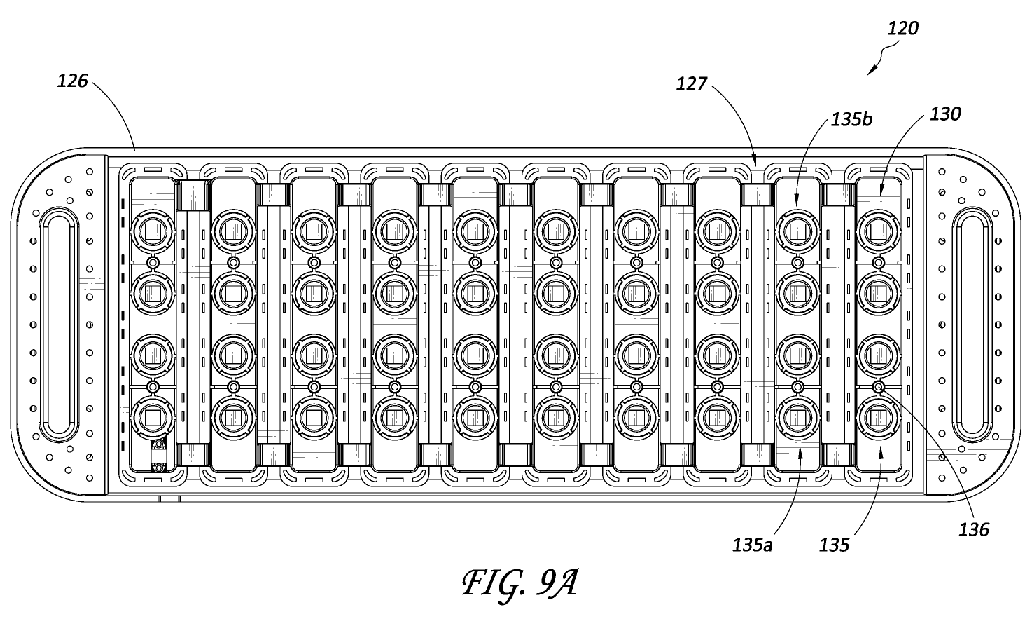

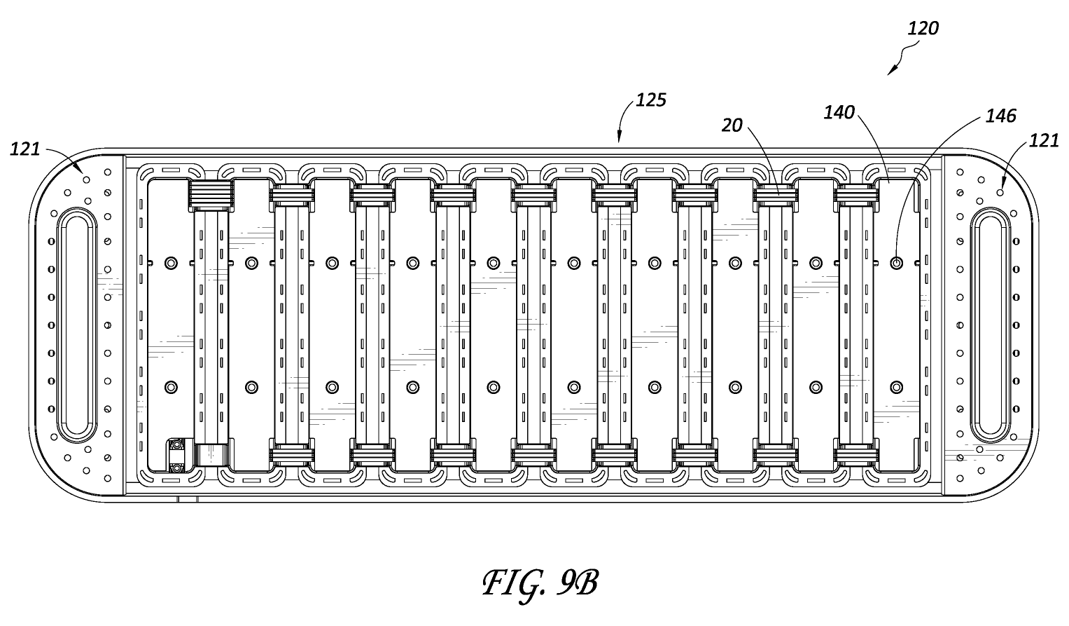

FIG. 9A illustrates an interior view of the front portion 120. As shown, in certain implementations, the peripheral edge 126 surrounds the body of the front portion 120. The base strips 130 can be seen mounted (e.g., nested) in the front portion 120 between a plurality of interior channels 127. In some embodiments, the plurality of interior channels 127 can align with the channels 124 and/or channels 184. The plurality of interior channels 127 can help separate the base strip 130 and facilitate the flexing/bending of the front portion 120.

The base strip 130 can include a plurality of housings 135 that can house one or more components of the light features 50. For example, the base strip 130 can include a plurality of round (e.g., circular) housings 135. The housings 135 can secure the LEDs in the front portion 120. In one example, the base strip 130 includes two pairs of housings 135 (e.g., a first pair 135a and a second pair 135b). Each pair can, for example, include two round housings. In one example, each pair can be positioned generally symmetrically (e.g., symmetrical about a longitudinal line extending through the center of the front portion 120) in the front portion 120, and/or secure the light features 50 therewithin.

The base strip 130 can include one or more openings 136. The openings 136 can be configured to receive a respective fastener 146 (e.g., screw, rivet, etc.). The fastener can secure a heat sink 140 to the base strip 130, as illustrated in FIG. 9B and discussed further below. In some variants, the base strip 130 is secured to the heat sink 140 in other ways, such as with an adhesive, press fit, snap lock, or otherwise. In certain implementations, the base strip 130 and heat sink 140 are integrated and/or are unitarily formed as a single unit.

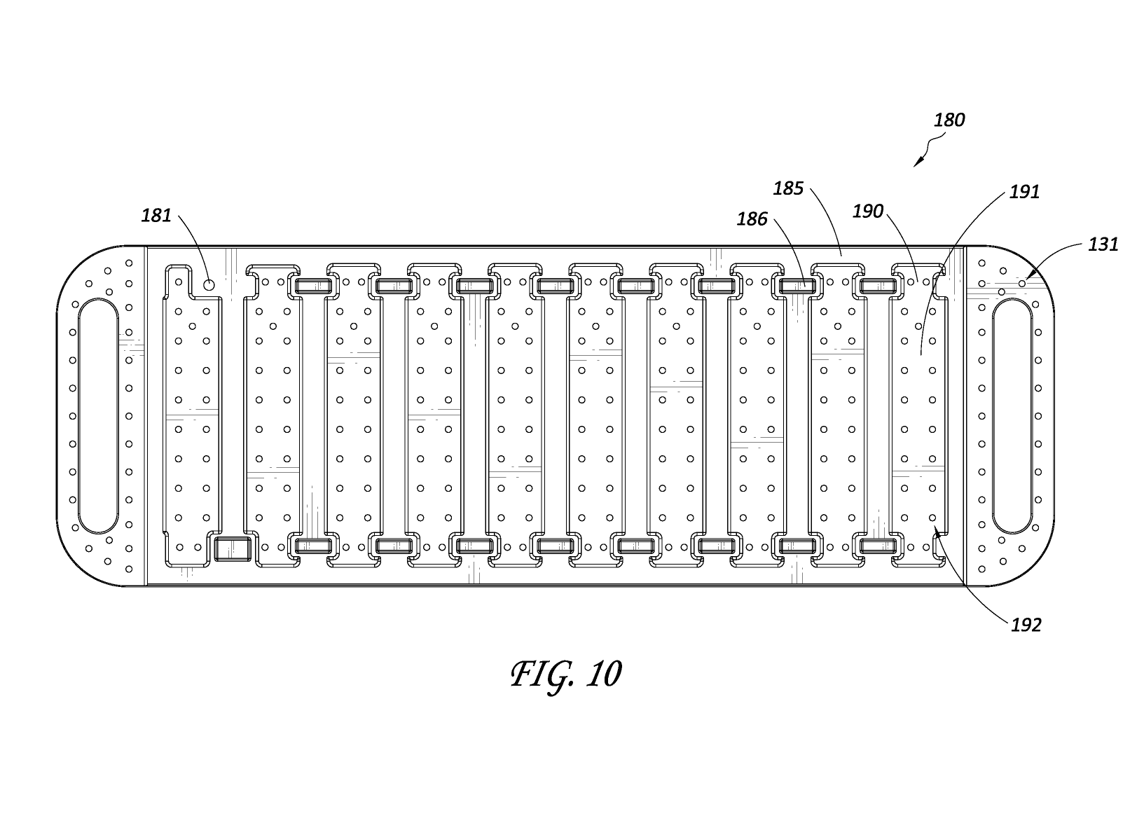

FIG. 9B illustrates another interior view of the front portion of the light therapy device 100. In this figure, heat sinks 140 are mated with the base strip 130 (the heat sinks 140 are not shown in FIG. 9A for purposes of presentation). The heat sinks can be manufactured from one or more materials (e.g., aluminum, aluminum alloys, copper, etc.) in different shapes. In some examples, each of the heat sinks 140 is a metal (e.g., aluminum) plate. The heat sinks 140 can be positioned adjacent to (e.g., behind, under, etc.) the light features 50 opposite a corresponding support strip (see strip 190 in FIG. 10).

The heat sinks 140 can collect (e.g., by conduction) heat generated from the light features 50 and allow the heat to be vented out. For example, in some implementations the heat sinks 140 can configured to supply to and/or vent through support strips 190 (see FIG. 10). The heat sinks 140 and/or support strips 190 can reduce the temperature of the light therapy device 100, thereby reducing and/or eliminating a need for an external cooling fan. Various embodiments of the device 100 have internal cooling elements (e.g., heat sinks 140 and/or support strips 190) and/or do not include an external cooling fan. Several implementations of the device 100 are configured to vent heat passively, such as without a fan. This can improve the energy efficiency of the device 100, reduce the total size of the device 100, and/or decrease noise during operation of the device 100.

In some examples, each row of base strip 130 includes a heat sink 140. The heat sink 140 in each row can be the same size and/or material as the heat sink 140 included in other rows, thereby facilitating the production and reducing the overall cost of the light therapy device 100.

Also illustrated in FIG. 9B is a plurality of openings 121 positioned along the interior surface of the front portion 120. The plurality of openings 121 can be configured to mate with and/or receive a corresponding projection 131 in the rear portion 180, as illustrated further in FIG. 10. This can facilitate securing the front and rear portions 120, 180 together.

Still referring to FIG. 9B, in some embodiments, the heat sinks 140 can be coupled to the front portion 120 with a fastener 146 (e.g., a pin, screw, etc.). The fastener 146 can include a head portion and a projection portion. The projection portion of the fastener 146 can be received in the opening 136, illustrated in FIG. 9A. The head portion of the fastener 146 can be pressed against and/or mate with a corresponding slot 191 (e.g., a recess portion) positioned on the support strips 190 (not illustrated in FIG. 9B, but see FIG. 10). In various embodiments, heat can flow from the heat sinks 140 through the fastener 146 to the support strips 190. The support strips 190 can be configured to inhibit heat from transferring through the silicone substrate of the rear and/or can direct heat out a side and/or rear of the light therapy device 100, thereby reducing the risks associated with buildup of excessive heat.

Also illustrated in FIG. 9B is one or more connectors 20, such as electrical couplings, wires, traces, etc. The light features 50 (see FIG. 7) can be communicatively coupled to one another and/or to an external power source with one or more connectors 20.

FIG. 10 illustrates an interior of the rear portion 180 of the light therapy device 100. As discussed above, the rear portion 180 can include the support strips 190. The support strips 190 can be manufactured from one or more materials. In some embodiments, the support strips 190 can be manufactured from materials that are generally harder than the rear surface 185 of the rear portion 180. For example, the rear surface 185 can be manufactured from a soft silicone-type material, and the support strips 190 can be manufactured from PC-ABS and/or other materials that can form a hard plastic. In some embodiments, the softer material of the rear surface 185 and the harder material of the support strip 190 can be manufactured through double-injection molding (also referred to as two-shot injection molding) whereby the softer and harder materials are combined into a single part within one molding cycle. Accordingly, the double injection molding of the support strips 190 into the rear surface 185 can provide the rear portion 180 with the strength of the harder material while maintaining softness, flexibility, and allowing the heat to dissipate through the support strips 190.

Still referring to FIG. 10, the rear portion 180 can include a plurality of recess portions 186 positioned between support strips 190. The recess portion 186 can positioned in the rear portion 180 and configured to receive a corresponding connector 20 of the front portion 120. This can allow the front portion 120 and rear portion 180 to press against one another more easily and/or reduce thickness, thereby contributing to the overall thin depth of the body 110.

The rear portion 180 can include the one or more projections 131, as mentioned above. The projections 131 can be received in the openings 121. The projections 131 can be aligned with the openings 121 such that when the front portion 120 is pressed against the rear portion 180 (or the rear portion 180 is pressed against the front portion 120), one or more projections 131 are received in the corresponding openings 121, thereby securing the rear portion 180 to the front portion 120. In some implementations, the support strips 190 can include one or more openings, which can mate with or be configured to receive projections 131, thereby securing the support strip 190 to the rear portion 180.

Also illustrated in FIG. 10 is the aperture 181. The aperture 181 can be a through hole. The aperture 181 can allow the passage of the connector 40, as discussed further below and illustrated in FIG. 11.

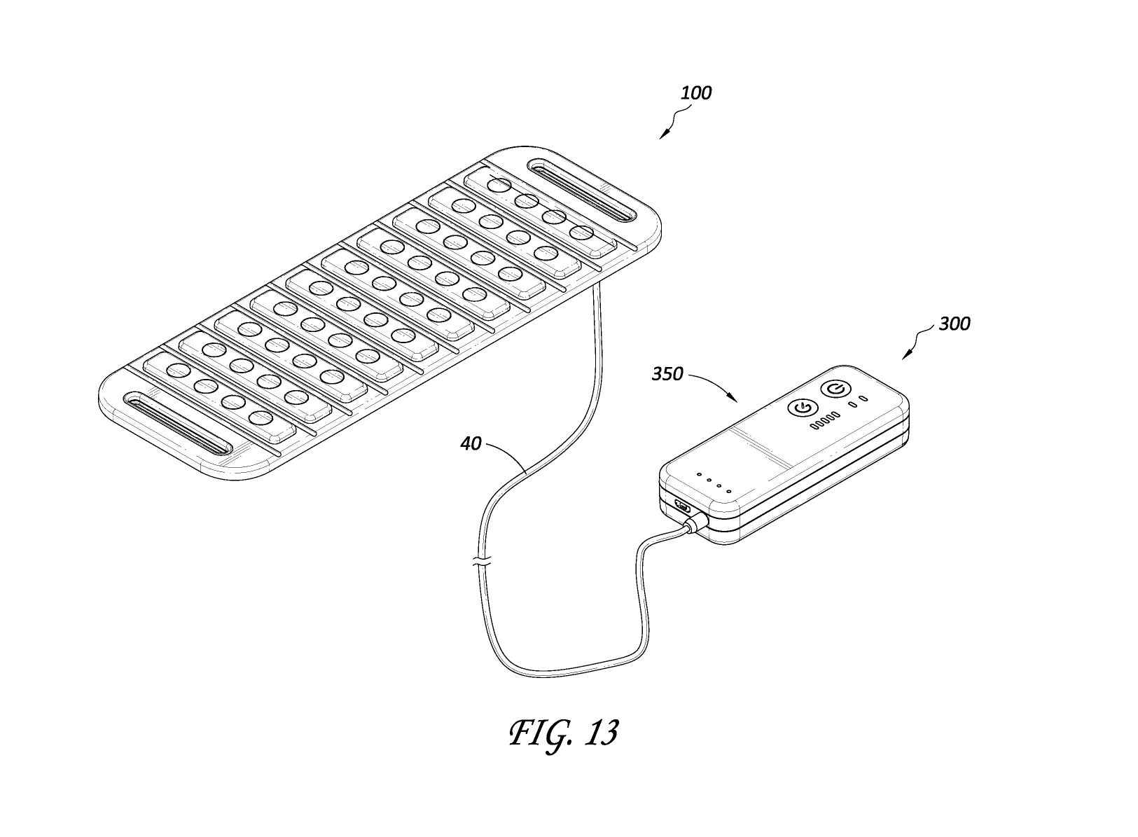

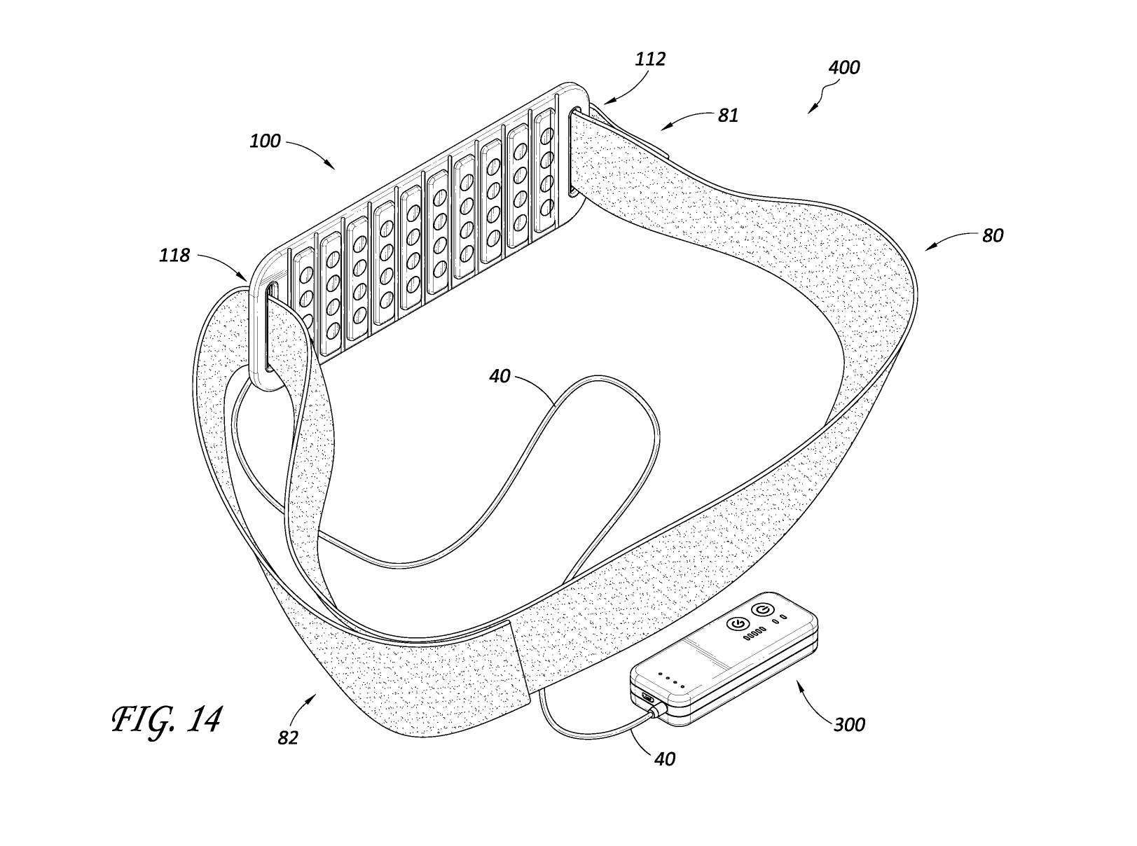

FIG. 11 illustrates an interior of the front portion 120 and rear portion 180 of the light therapy device 100. The connector 40 can be affixed to a receiving end 113 in the front portion 120. The connector 40 can pass through the aperture 181 and detachably connect to a controlling device (e.g., see controller 300 in FIG. 13). In some embodiments, the connector 40 can be a cable that communicates the flow of power (e.g., electricity) and/or low voltage communication (e.g., signal, data, etc.) between the light therapy device 100 and the controller 300.

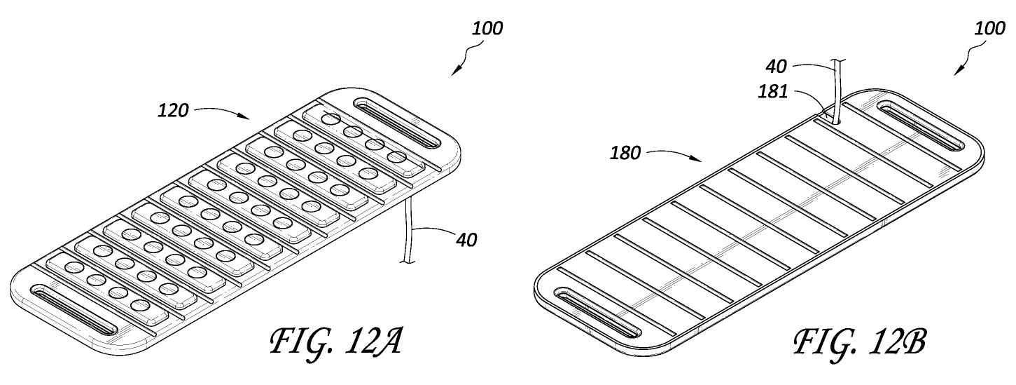

FIGS. 12A and 12B respectively illustrate top and bottom perspective views of the light therapy device 100, including the front portion 120 and the rear portion 180, in an assembled state. A portion of the connector 40 can be seen extending through the aperture 181 of the rear portion 180.

FIG. 13 illustrates the light therapy device 100 in use with a controller 300. The controller 300 enables a user to operate the light therapy device 100. The controller 300 can be communicatively coupled with the light therapy device 100. In some embodiments, the controller 300 can include a power bank to provide the necessary power for the light therapy device 100 to operate through the connector 40. As illustrated, in some implementations, the controller 300 is external to and/or remote from the therapy device 100. In certain variants, the controller 300 is integrated into, on-board, and/or part of the therapy device 100. For example, the controller 300 can be on the rear of the device 100.

The controller 300 can include a processor (e.g., an electronic processor and a memory) and a control interface 350. The control interface 350 enables a user to provide a command to the controller 300. For example, the user can actuate (e.g., press) a portion of the controller (e.g., a button) to enable the desired function. The control interface 350 can be configured to cooperate with the processor to control various operational aspects of the light therapy device 100. For example, the light feature 50 can be communicatively coupled to the processor of the controller 300, enabling a user to switch between different types of lights (e.g., red light, infrared light, etc.).

In some embodiments, the control interface 350 can include a timer. The timer can be communicatively coupled to the processor. The timer can enable automatic shutdown of the light therapy device 100. For example, the light therapy device 100 can be configured to deactivate (e.g., shut down) once the timer reaches a predetermined amount of time (e.g., 5 minutes, 10 minutes, 15 minutes, etc.). The control interface 350 can allow the user to switch between an on-state of the device 100 and an off-state of the device 100 upon activation of the control interface 350.

While various figures of the present disclosure illustrate the connector 40 in the form of a cable, the light therapy device 100 can be configured to operate without a cable. For example, the light therapy device 100 can be configured to communicate with the control interface 350 wirelessly (e.g., via Bluetooth® communication, Wi-Fi, electromagnetic induction, etc.). In some variants, the device 100 is configured to communicate with and/or be controlled by app or other software executed on a separate electronic device, such as a smartphone.

FIG. 14 illustrates a light therapy system 400, including the light therapy device 100 coupled to the controller 300 through the connector 40. A band 80 (e.g., a strap) can be seen extending through the first opening 112 and the second opening 118 to form a loop. The band 80 can be resilient and/or elastic. The band 80 can be manufactured from one or more materials (e.g., synthetic rubber, natural rubber, etc.). The materials used in the band 80 can be changed as needed to accommodate a user’s need. For example, the band 80 can be manufactured to be biocompatible, non-irritating, breathable, etc. to accommodate users with different preferences. In some embodiments, the band 80 can be configured to include a fastening system at each end. For example, the band 80 can include a hook-and-loop system at each end. In some embodiments, a first end 81 and/or a second end 82 of the band 80 can form a hook-and-loop system with the body of the band 80 to secure the ends of the band 80. In use, the band 80 can wrap around a body part of the user, allowing the light therapy device 100 to better conform with the body part.

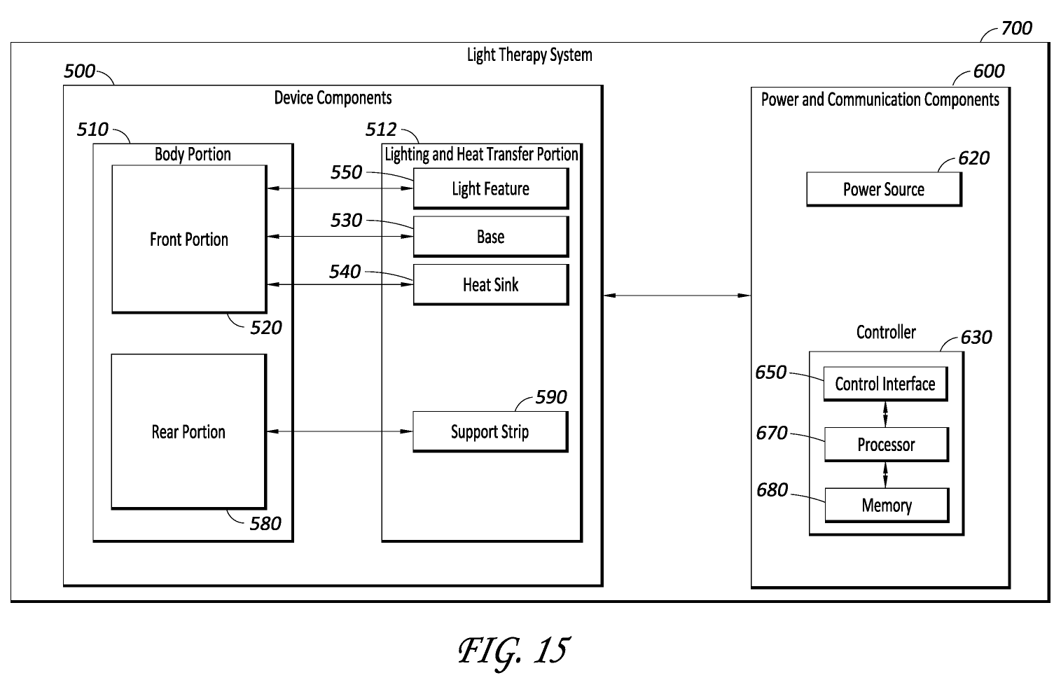

FIG. 15 schematically illustrates an embodiment of a light therapy system 700. The light therapy system 700 can include the same, more, or fewer components and/or features than those illustrated with respect to the light therapy system 400. In some embodiments, the light therapy system 700 can include device components 500, along with power and communication components 600. The device components can include a body portion 510, and a lighting and heat transfer portion 512. The body portion 510 can include a front portion 520 and a rear portion 580. The front portion 520 can include one or more features corresponding to the lighting and heat transfer portion 512. For example, the front portion 520 can include a light feature 550, a base 530, and a heat sink 540. The rear portion 580 can include one or more features corresponding to the lighting and heat transfer portion 512. For example, the rear portion 580 can include a support strip 560.

The device components 500 can be communicatively coupled with the power and communication components 600. The power and communication components 600 can include one or more power source 620 and a controller 630. The controller 630 can include a control interface 650, a processor 670, and a memory 680.

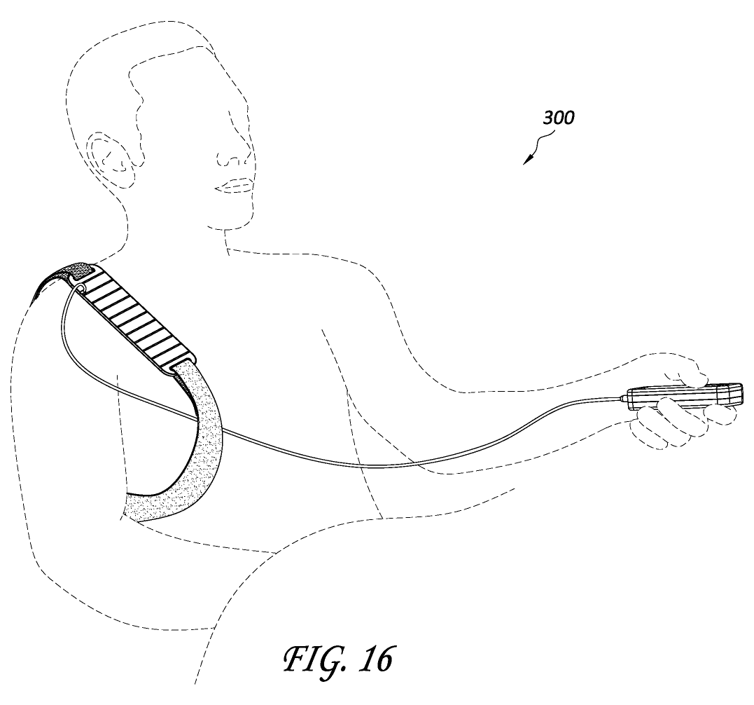

FIG. 16 illustrates an example of some components of the light therapy system 400 of FIG. 14 in use around an upper arm of a user. Although FIG. 16 illustrates the system 400 in use around the upper arm or shoulder, the system 400 can be used on other body parts (e.g., lower arm, hand, foot, leg, back, face, scalp, neck, etc.).

Certain terminology may be used in the description for the purpose of reference only, and thus is not intended to be limiting. For example, terms such as “front”, “rear”, “above”, “below”, “top”, “bottom”, “left”, “right”, and similar terms refer to directions in the drawings to which reference is made. Such terminology may include the words specifically mentioned above, derivatives thereof, and words of similar import. Similarly, the terms “first”, “second”, and other such numerical terms referring to structures neither imply a sequence or order unless clearly indicated by the context.

Conditional language used herein, such as, among others, “can,” “could,” “might,” “may,” “e.g.,” and the like, unless specifically stated otherwise, or otherwise understood within the context as used, is generally intended to convey that certain aspects include, while other aspects do not include, certain features, elements and/or states. Thus, such conditional language is not generally intended to imply that features, elements and/or states are in any way required for one or more aspects or that one or more aspects necessarily include logic for deciding, with or without author input or prompting, whether these features, elements and/or states are included or are to be performed in any particular aspect.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may permit, the term “generally parallel” can refer to something that departs from exactly parallel by less than or equal to 20 degrees. As another example, in certain embodiments, as the context may permit, the term “generally perpendicular” can refer to something that departs from exactly perpendicular by less than or equal to 20 degrees.

Some embodiments have been described in connection with the accompanying drawings. The figures are drawn to scale, but such scale is not limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Various embodiments and examples of light therapy devices, and associated systems and methods, have been disclosed herein. Although the invention has been disclosed in the context of certain embodiments and examples, it will be understood by those skilled in the art that this disclosure extends beyond the specifically disclosed embodiments to other alternative embodiments and/or uses of the embodiments and certain modifications and equivalents thereof. The scope of the present disclosure is not intended to be limited by the specific disclosures of preferred embodiments in this section or elsewhere in this specification, and may be defined by claims as presented in this section or elsewhere in this specification or as presented in the future.

What is claimed is:

1. A light therapy device configured to emit light to a body part of a user, the light therapy device comprising:
a front portion, the front portion comprising:
a first flexible substrate;
a plurality of channels positioned on an exterior of the flexible substrate;
a base strip embedded in the flexible substrate, the base strip being harder than the flexible substrate;
a plurality of lighting features in the base strip; and
a heat sink positioned adjacent to the plurality of lighting features, the heat sink positioned on an interior side of the front portion; and
a rear portion, the rear portion comprising:
a second flexible substrate;
a plurality of support strips positioned in an interior of the rear portion, the plurality of support strips being harder than the flexible substrate of the rear portion; and
a plurality of channels positioned on the exterior of the rear portion;
wherein the heat sink is configured to transfer heat generated by the plurality of lighting features to the plurality of support strips.

2. The light therapy device of claim 1, wherein the flexible substrate of the front portion and the flexible substrate of the rear portion comprise silicone.

3. The light therapy device of claim 1, wherein the base strip comprises PC-ABS.

4. The light therapy device of claim 1, wherein the rear portion is manufactured by double injection molding of the flexible substrate and the plurality of support strips.

5. The light therapy device of claim 1, wherein the heat sink is an aluminum plate.

6. The light therapy device of claim 1, wherein the light therapy device is configured to emit lights having a wavelength and frequency corresponding to red light.

7. The light therapy device of claim 1, wherein the light therapy device is configured to emit light comprising wavelengths associated with infrared.

8. A system comprising the light therapy device of claim 1 and a controller.

9. The system of claim 8, wherein the controller comprises a control interface and a processor to control an operation of the light therapy device.

10. The system of claim 9, wherein the controller is configured to control the operation including: an activation of the light therapy device, a predetermined time to deactivate the light therapy device, or a type of light that the light therapy device is configured to emit.

11. A method of providing light therapy to a body part of a user, the method comprising:
positioning a light therapy device over the body part, the light therapy device having a flexible body, the light therapy device comprising a first end and a second end, the first end including a first aperture and the second end including a second aperture;
positioning a band through the first aperture and the second aperture to form a loop;
securing the loop of the band with the body part of the user;
activating the light therapy device;
enabling the light therapy device to emit light to the body part of the user, the light corresponding to a predetermined frequency; and
deactivating the light therapy device after a passage of a predetermined time.

12. The method of claim 11, wherein activating the light therapy device comprises providing one or more commands to a controller through a control interface.

13. The method of claim 11, wherein a step of deactivating the light therapy device is performed by a controller after the passage of the predetermined time.

14. The method of claim 11, wherein the band is an elastic band comprising a first end and a second end.

15. The method of claim 14, wherein securing the loop of the band with the body part includes coupling the first end and the second end of the elastic band with a hook-and-loop system.

16. A light therapy device, comprising:
a body comprising a flexible portion and a hard portion, the body configured to conform with a body part of a user;
a plurality of channels positioned on the flexible portion, the plurality of channels configured to enable the body to flex; and
a plurality of light features configured to emit light to a body part of the user, the plurality of light features arranged to form an array of lights including a series of rows and columns, the rows arranged along a lateral orientation of the light therapy device and the columns arranged along a longitudinal orientation of the light therapy device, the array of lights configured to emit light to the body part uniformly.

17. The light therapy device of claim 16, wherein the array of lights includes ten rows and four columns.

18. The light therapy device of claim 16, wherein the body comprises a front portion configured to mate with a rear portion.

19. The light therapy device of claim 16, wherein each of the plurality of light features comprises a light-emitting diode and a lens, the light-emitting diode configured to emit red light.

20. The light therapy device of claim 16, comprising a plurality of heat sinks positioned adjacent to the plurality of light features.
